(19) United States
(12) Patent Application Publication
(10) Pub. No.: US 2014/0130806 A1
(43) Pub. Date: May 15, 2014

(54) SIDE-SPECIFIC FLUID-RETAINING CONDOM SYSTEM WITH A HOLDING PLUG

(71) Applicant: Leon B. KASSMAN, Rockville Centre, NY (US)

(72) Inventor: Leon B. KASSMAN, Rockville Centre, NY (US)

(21) Appl. No.: 14/045,826

(22) Filed: Oct. 4, 2013

Related U.S. Application Data
(63) Continuation-in-part of application No. 13/031,787, filed on Feb. 22, 2011, now abandoned.
(60) Provisional application No. 61/338,421, filed on Feb. 19, 2010.
(60) Provisional application No. 61/342,815, filed on Apr. 20, 2010.
(60) Provisional application No. 61/404,865, filed on Oct. 12, 2010.

(51) Int. Cl. A61F 6/04; A61F 6/06

(52) U.S. Cl. CPC A61F 6/04; A61F 6/065
USPC 128/844

(57) ABSTRACT

A condom assembly includes a plug and a condom. The condom has an elongated shape with an inner and an outer surface is provided. A barrier or strut is formed on the condom which prevents any liquid exchange between the inner and outer surfaces during the use of the condom. The plug is disk shaped and has a circumferential groove receiving the bead of the condom. A sealing member is provided that forms a seal in the groove between the plug and the bead of the condom. A cover may be provided to fit over the plug. Alternatively, an assembly is provided with a condom and a cover, the plug being omitted.

### RELATED APPLICATIONS

This application claims priority to U.S. patent application Ser. No. 13/031,787 filed Feb. 22, 2011, which in turn claims priority to the following provisional applications: Ser. No. 61/338,421 filed Feb. 19, 2010; Ser. No. 61/342,815 filed Apr. 20, 2010 and, Ser. No. 61/404,865 filed Oct. 12, 2010, all incorporated herein by reference

### BACKGROUND OF THE INVENTION

A. Field of Invention

This invention relates generally to methods of contraception and prophylaxis against diseases transmittable by sexual contact. More particularly, the invention concerns a new condom system consisting of several distinguishable novel improvements of a conventional male or female condom, including providing several novel means of providing a barrier between the inner and outer surfaces or sides of the condoms. All improvements were created in order solve the problems created by conceptualizing condoms from a perspective different from the way they have been previously conceived. In one aspect of the invention, a plug is used as vehicle for a condom during storage and shipping, with sealing elements being provided on the plug to seal the inside surface from the outside surface of the condom.

B. Description of the Prior Art

A condom (typically made from a non-porous material, including synthetic rubbers, latex, polymers, co-polymers and sheepskin) is a commonly used and generally effective barrier method used by either partner to provide complete protection against transfer of bodily fluids from one partner to the other. Such devices are effective for prevention of transmission of STDs and contraception as long as the physical barrier remains intact. In some cases however, the condom or the like may develop microscopic leaks or may contain small and perhaps unnoticeable perforations which can permit the transfer of microorganisms or of sperm across the barrier. In other cases the condom may rupture and permit substantial transfer of bodily fluids with the consequence of possible infection or conception. Although a complete rupture of a condom is relatively rare, it has been found that leaks sufficient to permit the escape of microorganisms in infectious amounts are by no means uncommon. In fact, the AQL (quality limit) posted by the FDA is 0.04 meaning that due to the imperfections known to exist in all latex condom manufacture, a very few of each lot that actually passed all FDA sampling testing is acknowledged by them that they will fail during regular use. Accordingly, the emergence of more serious and even fatal STDs such as AIDS has caused concern that even relatively reliable total barrier methods may not be satisfactory. Therefore, included in the preferred embodiment of present invention is a unique means to provide a second, chemical, barrier or treatment behind the barrier of the condom, on the male side of the condom, where the chemical has been demonstrated to kill nearly all pathogens, on contact, including HIV/AIDS.

In the past condoms have been thought as a product in and of itself whose purpose was to be donned onto the penis to serve as a barrier against the transmission of diseases during vaginal, anal, or oral sexual encounters to, or from either partner. Similarly, condoms have been used on various inanimate devices and objects to add a measure of safety and cleanliness when the devices are used for stimulation. However existing condoms are not satisfactory for several reasons.

A specific need exists in as much as the preponderance of the sexual transmission of disease with percentages that may exceed 60% are in the direction from males, infecting, then non-infected females, or during male to male sexual episodes then non-infected males or the opposite, or even when the male may inadvertently re-infect and already infected female, and while condoms alone serve as the only known means of stopping the spread of these life threatening and certainly life altering diseases, and also serve as an excellent form of contraception, and since condoms break, or tear, or come off during sex, or may be removed by men at one time or another during sexual activities, it would be best, considering the above, if chemicals that could kill ambient viruses in semen or pre-ejaculate and de-activate sperm and/or remain on the surface of the males penis or in any folds there-on, therefore available to dose the penis but be restricted from the full and complete dosing of the interior of the vagina.

### SUMMARY OF THE INVENTION

A condom constructed in accordance with this invention includes an envelope made of pliable material and having a separate inner surface (part actually/ordinarily in contact with the penis when the condom is installed or donned in accordance with the recommendations of manufacturers), an outer surface (part that comes into contact with vaginal, oral, or anal surfaces), closed-end (part that acts as a repository of ejaculatory fluids) and bead-end (part containing the factory intra-rolled portion used to roll down and cover the shaft of the penis). The inner surface is hereafter called the male-side, and the outer surface is hereafter called the female-side of the condom.

The reason for distinguishing between parts of the condom becomes necessary in order to understand how the invention is actually a new condom system of interacting modifications that are required so as to achieve the various specific objectives of the invention. One objective is to isolate the male-side from the female-side during use and to maintain treatments (fluids or powders) that are introduced to the male-side, using a number of different means, from migrating from that side during manufacture, storage, or use. Similarly, the system enables the isolation of treatments to the female-side from similar migration or leakage.

The purpose of the treatments is to augment and extend the effects of the barrier provided by the latex or other elastomeric film itself. An alternative method for preventing the transmission of sexually transmitted diseases is to kill or inhibit the pathogenic microorganisms that may be in semen and vaginal secretions so that they are incapable of infecting the partners tissues, thus causing disease. While intra-vaginally placed spermicides have been used for contraception, alone or in combination with barrier methods, antimicrobial materials have not been approved for said use to prevent STDs. This may be because many of such materials have been shown to irritate internal tissues. Some prior art references, such as, Howett et. al. (U.S. Pat. Nos. 6,192,887, 6,458,346, 6,635,242) and Bergeron et. al. (U.S. Pat. No. 7,192,607) have demonstrated the effectiveness in both laboratory and actual clinical trials of the identical compounds differentially called sodium dodecyl sulfate (SDS) and sodium lauryl sulfate (SLS). The FDA, along with most chemists consider both names to be an accepted manner of expressing the same number (25) of carbon atoms in the compounds chemical structure. It is important to make note that SDS is one of the most often used chemical ingredients used regularly and commonly by man and, indeed, the compound has been on the FDA's GRAS (Generally Recognized As Safe) listing of chemicals automatically considered safe for humans to use. The present invention uses this chemical as one active ingredient that can function as a microbicide, although by no means is the invention restricted to being used solely for that chemical.

The art is rich with inventions that focus on generating pleasure for women by introducing nubs, ridges, lines and extra material onto the condom body with the additional an concomitant purposes of reducing slippage and leakage. In addition to one product called Inspiral™, the inventions discussed in U.S. Pat. No. 6,074,671 (Oldham), U.S. Pat. No. 6,298,852 (Michelson), U.S. Pat. No. 6,983,751 (Osterberg), U.S. Pat. No. 5,954,054 (Johnson), U.S. Pat. No. 6,135,112 (Strauss), and U.S. Pat. No. 5,715,839 (Strauss), WO 97/30668, Design Pat. No. 420,127, U.S. Pat. No. 6,308,708, Design Pat. No. 252,949, Design Pat. No. 448,471 are particularly noteworthy for background. However, the present invention provides novel improvements not foreseen by these earlier inventions.

To achieve and maintain the treatments to the separate sides, the treatment itself is introduced either directly at the factory to the condom itself, or maybe introduced just before use by means of special applicators that are packaged along with the condoms. To maintain the treatments post their introduction to the condom, regardless of when and how they are introduced, certain structural changes in the part of the condom ordinarily covering the shaft of the penis are part of the invention. These structural changes not only retain the treatments but act simultaneously to maintain erectile turgidity by restraining the normal outflow of ambient fluids that filled cavernosal chambers causing the erection while stimulating internal and external vaginal surfaces in a manner that cause additional excitement.

A further feature of the invention is a unique and novel condom-system, which in various iterations may include a reconfigured condom, a detachably attached plug that fits within the bead-end, at least one or several thickened circumferential rings of same material at or toward the condom bead-end and which extend both inward and outward at that point to constrict (male) and stimulate (female) anatomical parts, and one or more types of applicators used variously to orient the condom and hold and/or provide a means of treating the male-side of the condom with any fluid/powder and to also act to hold such treatment from migrating to the opposite side of the condom during manufacture, storage, or use.

The purpose of retaining the treatment on the side to which it is introduced, and by such retention, prevent or limit to the extent possible, if not 100%, is determined by the prescribed use and/or knowledge about that treatment. It is acknowledged that some treatments that may be inert to one sex are not well tolerated or can contribute/cause potential adverse events, such as abrasion in certain members of the opposite sex. By preventing or limiting the cross sexual effect of a treatment additive that may migrate through inadvertant means from the administered side to the migrative side is one of the purposes of this invention and patent application.

This patent application further extends Kassman's Drug Delivery Package U.S. Pat. No. 7,617,827 that was awarded on Nov. 17, 2009. That Patent substantially covers a means of adding a medicinal substance, particularly to the outside of a conventionally fabricated condom, made from any elastomeric product, by using a specially constructed/pre-packaged applicator at a time very close in proximity to the time the condom is actually being used, and keeping that medicinal substance in a pouch or container that maintains the substance out of direct contact of the condom until the time just before use.

The literature has provided for devices, used for other purposes, to be attached to or detachably-attached to condoms. Schroeder U.S. Pat. No. 4,726,359 described ‘end-caps on rolled condoms that were meant to indicate the direction of unrolling the condom and to maintain sterility. Since these caps fit over the whole condom and are not, as the present patent maintains, disks inserted into the condom to prevent migration of fluids, this patent is not persuasive over the present application. Foldesy U.S. Pat. No. 5,163,448 mentions the use of a ‘rolled-ring’ in connection with the application of a medicament to a condom. The present invention is round but not at all a ring, but, instead, a solid round disk, And, it is not used to disperse lubricants but, instead, to hold any previously dispersed lubricant or microbicide in the same position that it was originally dispersed into. Also described is a ‘plug’. Here, too, the described plug acts as a reservoir, a container, holding a medicament or other material but, in no way serving as a block against migration of any lubricant. Jones U.S. Pat. No. 5,316,019 describes a ring member and a tolled-up condom but the ring is exactly that, a ring, used as a means of applying the condom with said ring being frangible therefore allowing it to expand as it is drawn over a penis diameter presumably larger than the diameter of the ring at rest. From the description, the ring and the condom are fully immersed in the included lubricant and therefore the ring does not serve as a means of preventing lubricant migration. Kuyumciyan U.S. Pat. No. 5,471,998 and Steer U.S. Pat. No. 5,531,725 describe tubular applicator devices, one a hollow tube and the other a strip of adhesive tape with the purpose of improving condom application or condom retention on the penis but neither device mentions preventing lubricants from migrating from one part of the condom to another.

The current patent application focuses primarily on the inside surface (male-side) of the male condom and by extension, will also apply to the male-side surfaces which exist opposite from the interior (female-side) surfaces of a female condom once it is inserted into the vagina.

The current patent shall cover natural rubber latex, polyethylene, polyisoprene and any other polymer which may or may not have elastomeric properties permitting even the most limited amount of stretch, or to compositions and configurations which may be constructed of any substance (of less or no elastomeric properties) so as to form a tubular shaped configuration roughly open at one end and closed of the same, where the total device is made of a single substance, or of other substances that can be bound to the substance used to form the preponderance of the device. The current patent may also be used as an ‘over-condom’, a device that is donned just like a standard condom but, by choice, it is donned either over a standard condom possibly for additional protection, or it is donned over any device that had already been donned by the user of the user's partner to the user, prior to intercourse, for any benefit or purpose then desired by the user. The device may be used for such purposes as contraception, prevention from disease, support, or the means of dispersal of any medicinal product directly to the penis and/or to the vagina.

Condoms, predominantly male condoms, but female condoms as well, have been designed largely to conform to the roughly tubular or somewhat triangular configuration (see Kassman patent application Ser. No. 10/016,219 Hermaphroditic Condoms) and fabricated from a substance that will, or configured in a manner to essentially fit themselves, post donning, to the physiological extent or confines of either an erect penis or to largely expand toward and to the walls of the vagina.

To accommodate human anatomic differences these devices have be made from mostly some kind of substance that not only has elastomeric properties, permitting some form of stretch, but also of substrates that are significantly impervious to the passage of sperm and similarly impervious to the passage of many or most bacteria, viruses, fungi, or other pathogens that cause almost any type of Sexually Transmitted Infections (STIs), as well as the transmission of those infections mostly associated and which mostly originate in women's vagina like vaginitis. The current patent application's extent is meant to include sheaths of all types designed to prevent the transmission of any unwanted, naturally occurring, or other condition whether it be infectious to the opposite partner or not.

Associated with the development of protective sheaths (regardless of whether they are male or female condoms or condom like structures), has been a parallel development or use of naturally occurring products or combinations of products whose lubricious nature could be used to more easily and comfortably allow an erect male penis to be inserted into a vagina. Although many women's vagina's have natural means where substances are emitted that act to lubricate the internal tissues, some women do not, and there are instances where such lubrication is impossible, truncated, or non-existent. To overcome these circumstances men and women have been using many different substances and methods to provide more comfort during intromission. These substances have included bees wax, lard, butter, grease, olive oil, whale oil, and any other substance that offers some slippery-ness during use including saliva.

Eventually, through advances in technology, substances were developed that were highly lubricious, maintaining that characteristic for years (when sealed an-aerobically in plastic or foil condom packages) so that condoms could be used with more comfort. Allied to this development was the discovery that all petroleum based oil products, and many naturally based oil products, were not recommended for use—especially never with natural rubber latex substrate products) because, over time, and relatively quickly, the petroleum oil acts to interrupt the natural rubber latex chemical bonds with the net result of weakening the surface therefore compromising the ability of the latex to serve as a barrier to any pathogens.

When water based lubricants and those that used silicone as a medium were discovered and implemented, the problem concerning disintegration of the latex bonds was ended and, today, all lubricants used in the natural rubber latex products are either water-based lubricants or silicone based lubricants—as now required by FDA statute. Water is also an excellent medium to dissolve many ingredients. Therefore, the current patent envisions the use of a water based lubricant on the female-side of the invention and, additionally, the use of water but not as a lubricant to be the medium into which a microbicide or spermicide is dissolved and then variously introduced to the male-side of the invention either in the factory or by virtue of an applicator or a frangible pouch shortly before or during the actual donning of the condom and before actual use during any form of sexual contact between individuals or between an individual and a device used for stimulation (masturbatory use).

Other elastomeric products, but not fabricated from natural rubber latex, do not share that chemical deterioration problem, and, therefore, there is no proscription about the use of petroleum or natural based oils for them, except where the manufactures of those oils indicate that they should not be used openly on the skin and particularly not on parts of the skin where those tissues will be adversely affected.

One characteristic of silicone lubricants is that they tend to spread from molecule to molecule permeating across almost all non-absorptive surfaces by means of capillary action. This characteristic is used in the manufacture of all conventionally sealed latex condoms. Drops of the lubricant are added immediately after the time the already inter-rolled condom drops on to a foil laminate band and prior to the other half of the laminate is joined and sealed and then crimped and cut into the roughly 2″×2″ squares commonly available and seen as conventional condom packages.

Ordinarily, the position of the condom that dropped onto the foil is not routinely controlled and therefore when sitting on the foil waiting for the drops of lubricant, they may be oriented with their closed end up or, just as commonly, oriented with their open end up. Therefore, the lubricant may drop into the interior portion (male side) of the condom or it may drop on the exterior portion (female side) of the condom. While care may be taken by manufacturers in the attempt to ‘target’ a drop or multiple drops of said lubricant onto or into the near center of a condom (regardless of its own orientation) the lack of precision, every time, placement results in some lubricant finding its mark at the edge of the condom, or on its bead, and not in its center. The result is that there is more than occasional spillage of the lubricant onto the outside (here referred to as ‘female-side’) of the condom. Manufactures rely on the natural capillary action of the lubricant to spread itself, even throughout the inter-rolled portion of the condom, over time. This is the conventional method used in the industry and to make certain that the correct amount of lubricant with sufficiently limited viscosity was used, so that the lubricant will ‘flow’ into all condom recesses and folds. The FDA requires that a certain percentage of the lubricant should be spread over the entire surface of the total condom (interior and exterior/male side and female side) and regularly tests to make certain that this condition applies.

The spread of the lubricant on all surfaces of the condom the lubricity of the lubricant is a concern because manufacturers do not want the condom so slippery as to easily or immediately allow the condom to be dislodged off the penis during coitus. The present invention strictly controls the surfaces treated and migration of the lubricant.

With the exception of the Frank Sadlow (U.S. Pat. No. 5,857,466) patented ‘multi-size’ (girth and length) condoms, most condoms are made on standard (straight walled or contour shaped) dipping mandrels and are of a uniform width (diameter) particularly for the part of the condom commonly used to cover the shaft of the penis. Thus, measuring form the open (bead) end of the condom the first 3″ to 5″ of the condom is configured to compressively hold itself around the erect penis width sufficient foot-pounds of compressive force to overcome the effects of the lubricant. In fact, many condoms are designed so that they begin to flare out around the glans of the penis starting approximately 4″ to 5″ from the same bead (open) end with such flare ending in the reservoir tip that would ordinarily be positioned over the tip of the glans end of the penis. Therefore, in this area, the lubricant, that had deposited itself there serves to make the folds of the extra, flared, portion of the condom, during coitus, more easily move over the glans of the penis therefore achieving the designed for additional frictional sensation and, hoped for, satisfaction from using that ‘expanded’ form of condom product.

Although there are variations of this, as packaged, by different manufactures, essentially, this is the state of affairs currently. If an individual wants to add lubrication, whether to the inside or the outside of the condom, in addition to the ambient lubrication already included, there are a myriad of water-based or silicone-based products, consistent with the chemical composition of the natural rubber latex, that are available and which are commonly used.

It is an object of the present invention to provide a male condom that is designed and constructed to be highly effective in preventing unwanted pregnancy as well as combating public health concerns, such as the transmission of STDs through the exchange of bodily fluids during sexual activity.

The resulting condom system has several advantages such as:

1) To provide a male condom that is easy to properly apply to the penis.

2) To provide a male condom that is securely retained on the wearer's penis during sexual activity.

3) To provide a male condom including a microbicide/spermicide or other medicinal chemical.

4) To apply the microbicide/spermicide medicinal chemical to the interior (male side) of the condom and to retain it (the preponderance of it) there during coitus.

5) To automatically (by virtue of the act of donning) disperse the microbicide/spermicide from a reservoir contained within the condom or within an applicator attached to the condom at the time of use.

6) To introduce said microbicide/spermicide at the time of the manufacture or by the user at the time of use, in a manner so as to maintain said chemical on the male side of the condom using either physical/mechanical and/or physical/mechanical/chemical barriers and to delimit its migration to the female side of the condom and to delimit its migrating into the vagina during coitus.

7) That, notwithstanding the uniformly rounded, cross-section profile of formed condom rings as described by Strauss in U.S. Pat. No. 5,715,839, the present invention specifically forms thick rings of highly articulated shape that are designed to retain the chemical microbicide/spermicide on the male side of the condom. The added thickness of the latex has the effect of adding compressive pressure to the penis resulting in retarding the outflow of ambient fluids that filled cavernosal sacks within the penis thereby contributing to the erection. By retarding outflow the thicker circumferential rings result in erectile maintenance. Furthermore, concurrently due their shape and thickness, and by virtue of specific added design, the reciprocal part of these same rings on the outside (female-side) of the condom shall act to further excited female anatomical parts that they may contact during coitus.

The information contained within U.S. Pat. No. 6,135,112 is illustrative of the current state of the art. It fails to provide for erectile maintenance preferring to advise against tightness as causing discomfort. That patent contains general language concerning slippage and leakage and similar language regarding clitoral stimulation. Other than the minimum number of rings and their relative spacing the patent is inadequate in foreseeing that fluids on the male side of a condom must be blocked by engineered structures not by simply arraying a number of similar sized dykes that are quickly and easily over-topped.

8) New System Condoms will not have the tendency to slip-off during coitus because lubricants (that lower the coefficient of friction) are restricted to the female-side of the condom. (Treatments to the male side will be in the form and type to either have null effect or, even to have an opposite effect.)

9) Female anatomical surfaces that may be irritated by certain spermicide or microbicides will be protected from exposure because said chemicals are retained on the male-side of the condom.

The new condom system offers a variety of way of treating the male-side of the condom with microbicides/spermicides.

One of the problems with microbicides is that they can contribute/cause toxicity in the vagina, along with abrasive changes in vaginal epithelial tissues. These changes potentially weaken the normal blocking power of skin type tissue (even internal lining are a kind of skin) by causing an topical abrasion—resulting in an easier pathway for infection than if the microbicide were not used. Some abrasions have been seen colposcopically during clinical trials.

Since treatments applied to the inside of the condom will, over time, migrate by capillary action to the outside of the condom the New Condom System isolates the microbicide to the male-side of the condom. This is accomplished by inserting a detachably attached plug into the bead end of the condom after the condom has been dosed during packaging. The plug consists of a roughly spherical structure which may or may not be solid or may or may not be a ring with its center void. At the periphery of the plug a concave groove is created to hold the finished/bead-end of any rolled condom. The plug is constructed of an elastomer plastic of sufficient rigidity to overcome the compressive forces of the attached condom without deforming. The dimensions of the plug may vary in accordance with the circumference of the condom finished/bead-end with the preferred embodiment being at least 5% greater in circumference than the attached condom so that the condom will hold onto the plug by compressive force. This dimension may be increased which will directly increase the compressive force so as to improve the grip of the condom and resist its dislodging prematurely. Additionally, such additional compressive force will further prevent any treatment introduced into the male-side of the condom from migrating or leaking out during manufacture, shipment, or shelf-storage. The grooved part of the plug is also referred to as its rim. The plug is designed with a finger hold central strut to permit it to be easily grasped and removed. Additionally, the top most edges of the plug are diminished in two areas further enabling the plug to be easily removed from the condom just prior to donning. The fact that the plug sits in the rolled up section of the condom orients the user as to the correct-way-on donning direction to achieve additional speed in donning as well as to assure that the condom is donned in the correct-way-on orientation so that it may be fully rolled onto the erection.

In another preferred embodiment the plug's center is removed leaving mainly the grooved rim to which a condom can be attached and held. In this iteration, the structure of the rim is increased in rigidity to compensate for the loss of its center strut. Such increase may be accomplished by changing the formulation to a more rigid form of plastic ingredient or by increasing the wall thickness of said substrate. The rim is further bifurcated (split into an upper section and a lower section) to which the condom is actually assembled. The bifurcated halves are further modified with screw threads enabling each half to be mated together with at least ½ turn. Further changes to the top half of the rim causes a partition designed to fit against the outer side of the finished/bead-end of the condom once it is attached in a manner that further compresses the finished/bead-end so as to provide a further barrier against the migration or leakage of any treatment that was introduced into the male side of the condom after the condom was assembled to rim. The rim may be used as a round applicator to aid in the positioning and donning of the attached condom. Once dosed, the top of the applicator is screwed down over the bottom half (to which the condom has been pre-factory applied) applicator in a manner so that an interior stop on the top cap presses against the side of the condom preventing any of the microbicide from leaking out during shipping or shelf storage. This is the same method used to prevent liquids from spilling out from plastic bottles if the bottle were inverted. Prior to use the user unscrews the top half and then uses the bottom half to position and then lower the condom with its contents held within) over the erection. This action dons the condom on to the penis with the final transfer being accomplished using the edge of the thumb to remove the condom from the applicator. Post application the applicator is discarded.

Another iteration uses a oblong applicator onto which a condom is factory mounted and into which, post such mounting, an amount of microbicide/spermicide is treated. The applicator is similar to the applicator described in Kassman U.S. Pat. No. 7,617,827 except that the top lip of the applicator was modified to better hold a snap cap made to fit over and snap down onto a condom that had been applied to the applicator. A snap-on cap is used to hold the microbicide fluid within the condom until the condom is used. The applicator is then used to squeeze open the condom, center it over the erection, and then to lower it over the erection. By thumb rolling the condom off the applicator it is then transferred to the penis and the applicator is discarded. Another iteration positions a frangible pouch/pellet tack-attached to the interior wall of the condom and further positions it below the internal rolled-up bead so as to roughly fall on the second two inches of the shaft of an erect penis or to start 2 inches from the point that the pouch/pellet begins measured from the fully unrolled finished/bead-end and extending for 1 additional inch toward the reservoir tip of the condom. The width of the pouch/pellet will be at least ¼ inch and its height above the wall of the condom to which it is attached will be at least ¼ inch. The pouch/pellet will be manufactured of a frangible material compatible with the condom substrate and which may be treated in a manner that will assure that its contents will be disgorged when the penis is inserted and the condom rolled down. The actual fracturing would be caused by rolling it down. To assist in this function the pouch/pellet may be pre-perforated.

Alternatively, the wall of the elastic condom, formed via dipping or heat sealing could be initially formed in a fashion so that a small self-material bladder is formed on the outside of the condom (which becomes the inside of the condom when it is striped off the mandrel. This bladder may or may not be filled with air and it may or may not be totally separated space from the wall of the condom on which it was formed. A secondary step would be needed to fill the bladder with fluid, seal it closed if were not already thus sealed and further create, as necessary, such weak points in the bladder's wall so that it easily ruptured either as the condom, then attached to the applicator were squeezed open or when the male's penis was thrust into the condom during donning.

Another iteration features a conventional condom configured on an applicator with a frangible pouch/pellet suspended just above the applicator in such a manner that when the apparatus is lowered onto the erection the penis itself may fracture the pellet. The pouch/pellet is permanently adhered to the applicator (as in the illustration) attached at the lateral sides of the applicator. When the applicator is squeezed open the lateral sides move in opposite direction causing the pre-perforated frangible pouch/pellet to fracture thereby disgorging the contents that were in the pouch/pellet into the male-side of the condom detachable-attached to the applicator below. The act of squeezing open the applicator in a manner during the moment that the applicator is being used to don the condom causes the pouch/pellet to be fractured thus releasing its microbicide/spermicide contents onto the penis. The action of inserting the penis into the condom using the applicator further drives and spreads the medicinal material from the then fractured pouch toward the business (reservoir-tip) end where it would best available to kill viruses.

The actual pouch/pellet would be of a very soft medical grade material—much softer and more fragile than a condom itself. The material used to hold the pouch/pellet in place would be compatible with the applicator wall to which it becomes applied. A cyanoacrylate or a rubber cement, or any other material commonly used to hold one type of elastomer to another without damaging the walls of either would be used.

For the iteration where the frangible pouch is actually attached to the wall of the condom, the material used to attach said pouch, and the number and configuration of the points at which such pouch is attached may be different with there being at least one such attached point. Similarly, the number and places as well as the means of attaching a pouch configured to be attached circumferentially to the inside of the condom may vary.

The structure of the frangible pouches may also vary in terms of their shape, the positioning of the frangible elements, the number and shape of the frangible areas or lines of frangibility may also differ. The position of the frangible lines may or may not influence the direction of the flow of the contents of the packages.

The material within any of the frangible pouches may be a spermicide, Or the material may be a microbicide. Or the material may be a substance which helps the condom better stick to the penis regardless of whether the penis was then wet or dry.

The material within any of the frangible pouches may stimulate the penis to achieve a greater erection. Or the material may be such to sustain the length of time that the naturally occurring erection is maintained. Or the material may decrease the sensitivity of the skin of the penis during initial or further friction particularly if the material was time reactive.

Alternatively, in the Condom Applicator model, the frangible pouch (possibly even thin coated paper that was perforated in advance, and then with another very thin coating externally applied post perforation to retain the medicinal product. That pouch could be attached to the sides of the applicator prior to the condom being also attached, so that it positioned itself, depending on the length of the same material loops that would be fitted over the applicator side holding areas, just within the inside of the then being attached condom.

The spreading of fluids is a necessary part of the new condom system. This is different from the initial dispersal. In dispersal, a fluid held within the pellet/pouch would, post rupture of the pouch membrane issue out from the pouch and mostly flow with gravity (therefore, momentarily, at least, toward the bead end of the condom but still on the inner side surface of the condom). This fluid may also be dried and be in the form of an extremely fine powder that would instantly mix with any ambient liquid/lubricant or with pre-ejaculate and with semen. If in liquid form, the flow outside over the open end of the condom, would almost immediately be impeded by the presence of the penis that either immediately post rupture is thrust into the condom as it is donned thru forming a kind of piston pushing the probably still gushing liquid toward the reservoir tip end and/or at least along the inside surface of the condom. Or, should the opening squeezing torque exerted on the applicator and transferred, at least, in part, depending upon the position of the pouch (if glued to the interior condom wall) or on the position of the pouch suspended and hanging freely between the interior sides of the condom and held in place by loops of same pouch material that had been detachably attached to the side indentions formed originally as part of the applicator, were NOT sufficient to fully fracture the frangible pouch, then certainly, the penis itself, as it it thrust into the condom would fracture the pouch membrane and immediately drive the then disgorging liquid or gel toward the reservoir tip end.

The goal of the above preferred iterations was to introduce a treatment into the male-side of the condom and retain it there. To help accomplish the patent further envisions that certain modification may be required to the condom itself. In addition to the iterations above, the bead end of the condom may be altered. The purpose of this alteration is an additional measure to further retard the migration of fluid treatment that was in the reservoir end and to further retard leakage of any fluid from the male-side.

A series of at least one internally (toward the penis) facing struts can be engineered to the dipping mandrel. These circumferential struts, cause by either engraving shaped, specially configured, indentations and/or extensions on mandrel farmers, so that when dipped the latex adhered and was formed into one of more circumferential struts. These struts may have single or multiple tiers, they may be rounded or comma shaped, and may be in any configuration that will enable the vulcanized rubber or elastomer substrate, once fully molded to be detached from the former. Additionally, the struts may be of a width and height of at least 1/16 inch and they may be uniformly shaped or have molded extensions to their peripheral configuration to that such extensions may protrude further in parts of the extension than at other parts of the extension. On example of the shape of the extension would be to create a continuous rib or ridge shaped like the aft part of a airplane wing. To allow for penises of different diameters, the thickness (width) of any two such ribs or the amount that they protrude from the condom wall may be different.

### BRIEF DESCRIPTION OF THE DRAWINGS

FIG. 1—Detachably-Attached Plug that can be used for mounting a condom in accordance with this invention;

FIG. 2—Condom detachably-attached to the plug of FIG. 1;

FIG. 3—Bifurcated Plug with top acting as compressive cap;

FIG. 4—Oblong Applicator with frangible Pellet/pouch;

FIG. 5—Penis fracturing pellet/pouch disbursing contents on to penis;

FIG. 6—Conventional condom with pellet/pouch applied to interior (male-side) surface;

FIG. 7—Anti-Leak condom extensions/grooves molded near to bead-end;

FIG. 8—Absorptive element added to inside surface between grooves;

FIG. 9—Frangible pellet/pouch attached to foil;

FIG. 10—Frangible/squeezable pellet used to inject treatment;

FIG. 11 shows an alternate embodiment of the plug of FIG. 1;

FIG. 12 shows another alternate embodiment of the plug with sealing elements for sealing the bead of the condom.

FIG. 13 shows a cross sectional enlarged view of the plug of FIG. 12;

FIG. 13A shows an enlarged detail of the ribs formed in the groove of the plug of FIG. 13;

FIG. 13B shows an alternate embodiment wherein O-rings are provided in the plug groove;

FIGS. 14A shows a cross-sectional side view of yet another embodiment of a condom assembly with a ring shaped plug and a somewhat rigid cover;

FIG. 14B shows a plan view of the ring for the assembly of FIG. 14A;

FIG. 14C shows a plan view of the cap for the assembly of FIG. 14A;

FIG. 15A shows orthogonal view of another condom assembly constructed in accordance with this invention;

FIG. 15B shows the assembly of FIG. 15A with the cover being removed;

FIG. 15C shows an orthogonal view of the cover from the assembly of FIG. 15A;

FIG. 15D shows an orthogonal view of the correctly assembled condom assembly; and

FIGS. 16A-16D show another condom assembly with a wedge-shaped cover.

### PREFERRED EMBODIMENTS OF INVENTION

The New Condom System offers a variety of way of treating the male-side of the condom with microbicides/spermicides.

One of the problems with microbicides is that they contribute/cause toxicity in the vagina, along with abrasive changes in vaginal epithelial tissues. These changes potentially weaken the normal blocking power of skin type tissue (even internal lining are a kind of skin) by causing an topical abrasion—resulting in an easier pathway for infection than if the microbicide were not used. Some abrasions have been seen colposcopically during clinical trials.

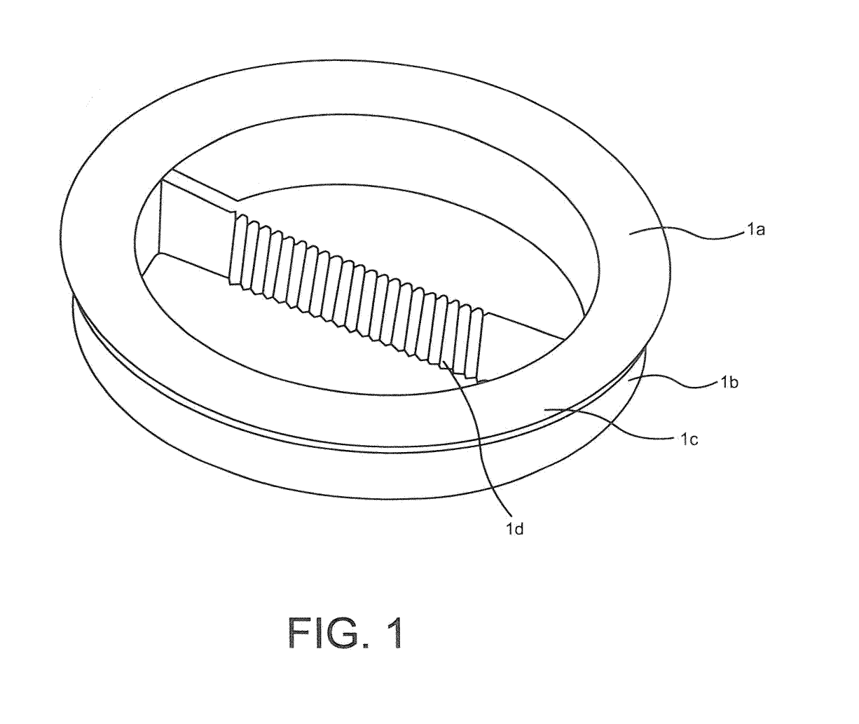

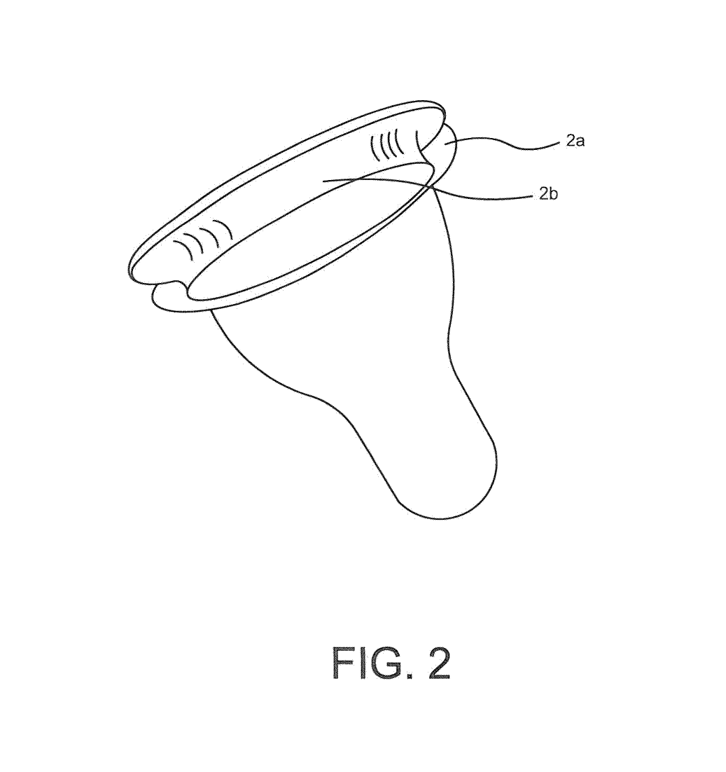

Since treatments applied to the inside of the condom will, over time, migrate by capillary action to the outside of the condom the New Condom System isolates the microbicide to the male-side of the condom. This is accomplished by inserting a detachably-attached plug (see FIG. 1 and FIG. 2) into the bead end of the condom after the condom has been dosed during packaging. The plug consists of a roughly disk-shaped structure (1a) which may or may not be solid or may or may not be a ring (1c) with its center void. At the periphery of the plug a concave groove (1b) is created to hold the finished/bead-end of any rolled condom (2a). The plug is constructed of an elastomer plastic of sufficient rigidity to overcome the compressive forces of the attached condom without deforming. The dimensions of the plug may vary in accordance with the circumference of the condom finished/bead-end with the preferred embodiment being at least 5% greater in circumference than the attached condom so that the condom will hold onto the plug by compressive force. This dimension may be increased which will directly increase the compressive force so as to improve the grip of the condom and resist its dislodging prematurely. Additionally, such additional compressive force will further prevent any treatment introduced into the male-side of the condom from migrating or leaking out during manufacture, shipment, or shelf-storage. The grooved part of the plug is also referred to as its rim (2b). The plug is designed with a finger hold central strut (1d) to permit it to be easily grasped and removed. Additionally, the top most edges of the plug are diminished in two areas further enabling the plug to be easily removed from the condom just prior to donning. The fact that the plug sits in the rolled up section of the condom orients the user as to the correct-way-on donning direction to achieve additional speed in donning as well as to assure that the condom is donned in the correct-way-on orientation so that it may be fully rolled onto the erection.

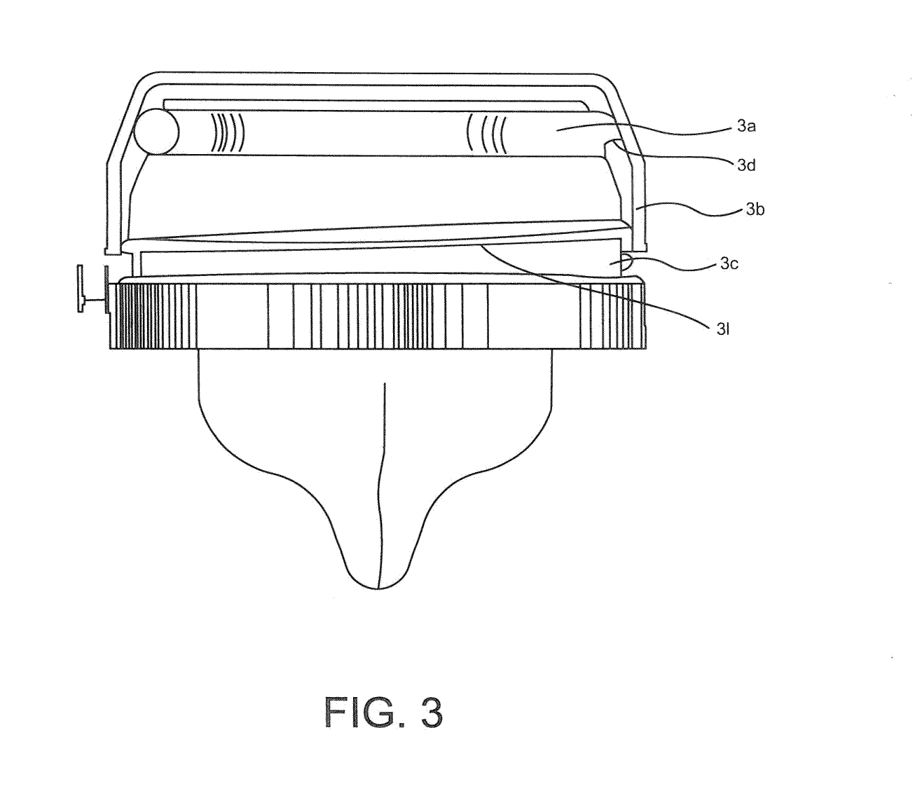

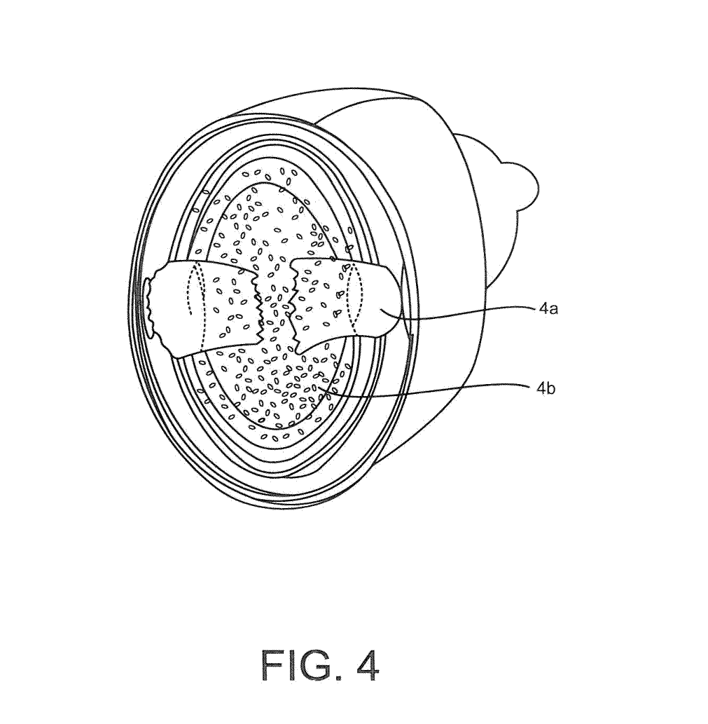

In another preferred iteration the plug's center is removed leaving mainly the grooved rim to which a condom can be attached and held (3a in FIG. 3). In this iteration, the structure of the rim is increased in rigidity to compensate for the loss of its center strut. Such increase may be accomplished by changing the formulation to a more rigid form of plastic ingredient or by increasing the wall thickness of said substrate. The rim is further bifurcated (split into an upper section and a lower section (3b, 3c) to which the condom (see 3d) is actually assembled. The bifurcated halves are further modified with screw threads (see 3e) enabling each half to be mated together with at least ½ turn. Further changes to the top half of the rim causes a partition designed to fit against the outer side of the finished/bead-end of the condom once it is attached in a manner that further compresses (3f) the finished/bead-end so as to provide a further barrier against the migration or leakage of any treatment that was introduced into the male side of the condom after the condom was assembled to rim. The rim may be used as a round applicator to aid in the positioning and donning of the attached condom. Once dosed, the top of the applicator is screwed down over the bottom half (to which the condom has been pre-factory applied) applicator in a manner so that an interior stop on the top cap presses against the side of the condom preventing any of the microbicide from leaking out during shipping or shelf storage (see 3a-e). This is the same method used to prevent liquids from spilling out from plastic bottles if the bottle were inverted. Prior to use the user unscrews the top half and then uses the bottom half to position and then lower the condom (with its contents held within) over the erection. This action dons the condom on to the penis with the final transfer being accomplished using the edge of the thumb to remove the condom from the applicator. Post application the applicator is discarded.

Another iteration uses a oblong applicator (4) onto which a condom is factory mounted and into which, post such mounting, an amount of microbicide/spermicide is treated. The applicator is similar to the applicator described in Kassman U.S. Pat. No. 7,617,827 except that the top lip of the applicator was modified to better hold a snap/screw cap (not shown) made to fit over and snap down/screw down onto or over a condom that had been applied to the applicator. A snap-on/screw down cap is used to hold the microbicide fluid within the condom until the condom is used. The applicator is then used to squeeze or hold open the condom, center it over the erection, and then to lower it over the erection. By thumb rolling the condom off the applicator it is then transferred to the penis and the applicator is discarded.

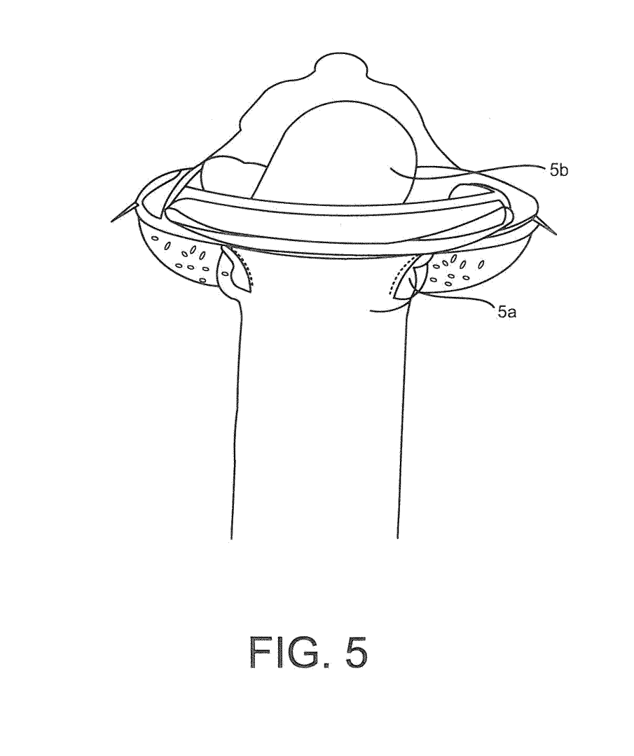

This iteration features a conventional condom configured on an applicator with a frangible pouch/pellet suspended (4a) just above the applicator in such a manner that when the apparatus is lowered onto the erection the penis itself may fracture the pellet (5 seen in FIG. 5). The pouch/pellet is permanently adhered to the applicator (as in the illustration) attached at the lateral sides of the applicator. When the applicator is squeezed open the lateral sides move in opposite direction causing the pre-perforated frangible pouch/pellet to fracture thereby disgorging (4b) the contents that were in the pouch/pellet into the male-side of the condom detachable-attached to the applicator below. The act of squeezing open the applicator in a manner during the moment that the applicator is being used to don the condom causes the pouch/pellet to be fractured (5a) thus releasing its microbicide/spermicide contents onto the penis (5a, 5b). The action of inserting the penis (see FIG. 5) into the condom using the applicator further drives and spreads (5b) the medicinal material from the then fractured pouch toward the business (reservoir-tip) end where it would best available to kill viruses.

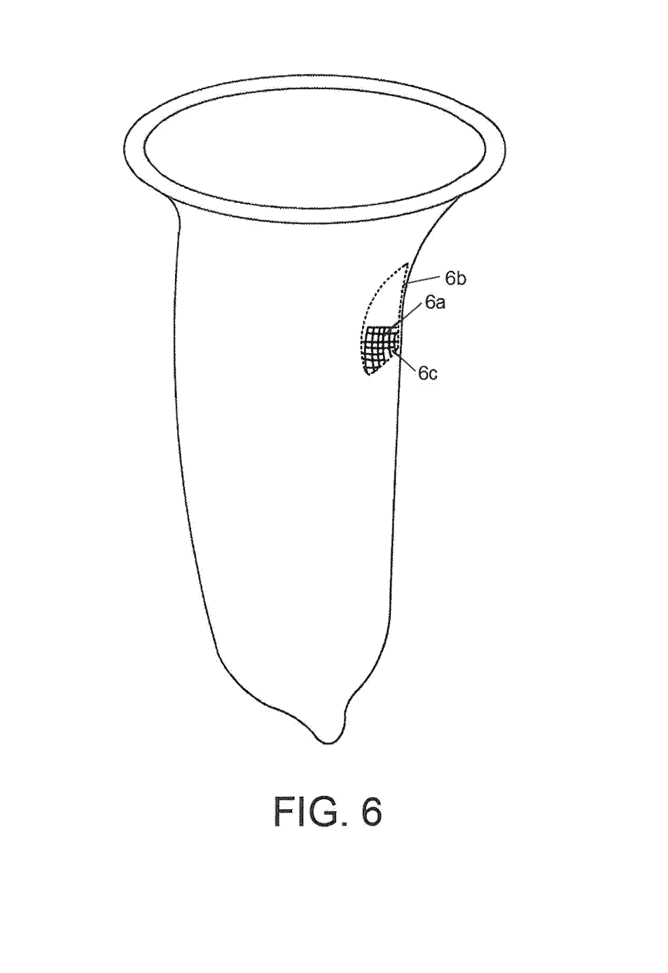

Another iteration positions a frangible pouch/pellet (6a in FIG. 6) tack-attached to the interior wall of the condom (see FIG. 6) and further positions it below the internal rolled-up bead so as to roughly fall on the second two inches of the shaft of an erect penis or to start 2 inches from the point that the pouch/pellet begins measured from the fully unrolled finished/bead-end and extending for 1 additional inch toward the reservoir tip of the condom. The width of the pouch/pellet will be at least ¼ inch and its height above the wall of the condom to which it is attached will be at least ¼ inch. The pouch/pellet will be manufactured of a frangible material compatible with the condom substrate and which may be treated in a manner that will assure that its contents will be disgorged when the penis is inserted and the condom rolled down. The actual fracturing would be caused by rolling it down. To assist in this function the pouch/pellet may be pre-perforated.

Alternatively, the wall of the elastic condom, formed via dipping or heat sealing could be initially formed in a fashion so that a small self-material bladder is formed on the outside of the condom (which becomes the inside of the condom when it is striped off the mandrel.) This bladder may or may not be filled with air and it may or may not be totally separated space from the wall of the condom on which it was formed. A secondary step would be needed to fill (see 6b) the bladder with fluid, seal it closed if were not already thus sealed and further create, as necessary, such weak points in the bladder's wall so that it easily ruptured either as the condom, then attached to the applicator were squeezed open or when the male's penis was thrust into the condom during donning.

The actual pouch/pellet would be of a very soft medical grade material (6c)—much softer and more fragile than a condom itself. The material used to hold the pouch/pellet in place would be compatible with the applicator wall to which it becomes applied. A cyanoacrylate or a rubber cement, or any other material commonly used to hold one type of elastomer to another without damaging the walls of either would be used.

For the iteration where the frangible pouch is actually attached to the wall of the condom, the material used to attach said pouch, and the number and configuration of the points at which such pouch is attached may be different with there being at least one such attached point. Similarly, the number and places as well as the means of attaching a pouch configured to be attached circumferentially to the inside of the condom may vary.

The structure of the frangible pouches may also vary in terms of their shape, the positioning of the frangible elements, the number and shape of the frangible areas or lines of frangibility may also differ. The position of the frangible lines may or may not influence the direction of the flow of the contents of the packages.

The material within any of the frangible pouches may be a spermicide, Or, the material may be a microbicide. Or the material may be a substance which helps the condom better stick to the penis regardless of whether the penis was then wet or dry.

The material within any of the frangible pouches may stimulate the penis to achieve a greater erection. Or, the material may be such to sustain the length of time that the naturally occurring erection is maintained. Or, the material may decrease the sensitivity of the skin of the penis during initial or further friction particularly if the material was time reactive.

Alternatively, in the Condom Applicator model, the frangible pouch (possibly even thin coated paper that was perforated in advance, and then with another very thin coating externally applied post perforation to retain the medicinal product. That pouch could be attached to the sides of the applicator prior to the condom being also attached, so that it positioned itself, depending on the length of the same material loops that would be fitted over the applicator side holding areas, just within the inside of the then being attached condom.

The spreading of fluids is a necessary part of the new condom system. This is different from the initial dispersal. In dispersal, a fluid held within the pellet/pouch would, post rupture of the pouch membrane issue out from the pouch and mostly flow with gravity (therefore, momentarily, at least, toward the bead end of the condom but still on the inner side surface of the condom). This fluid may also be dried and be in the form of an extremely fine powder that would instantly mix with any ambient liquid/lubricant or with pre-ejaculate and with semen. If in liquid form, the flow outside over the open end of the condom, would almost immediately be impeded by the presence of the penis that either immediately post rupture is thrust into the condom as it is donned thru forming a kind of piston pushing the probably still gushing liquid toward the reservoir tip end and/or at least along the inside surface of the condom. Or, should the opening squeezing torque exerted on the applicator and transferred, at least, in part, depending upon the position of the pouch (if glued to the interior condom wall) or on the position of the pouch suspended and hanging freely between the interior sides of the condom and held in place by loops of same pouch material that had been detachably attached to the side indentions formed originally as part of the applicator, were NOT sufficient to fully fracture the frangible pouch, then certainly, the penis itself, as it it thrust into the condom would fracture the pouch membrane and immediately drive the then disgorging liquid or gel toward the reservoir tip end.

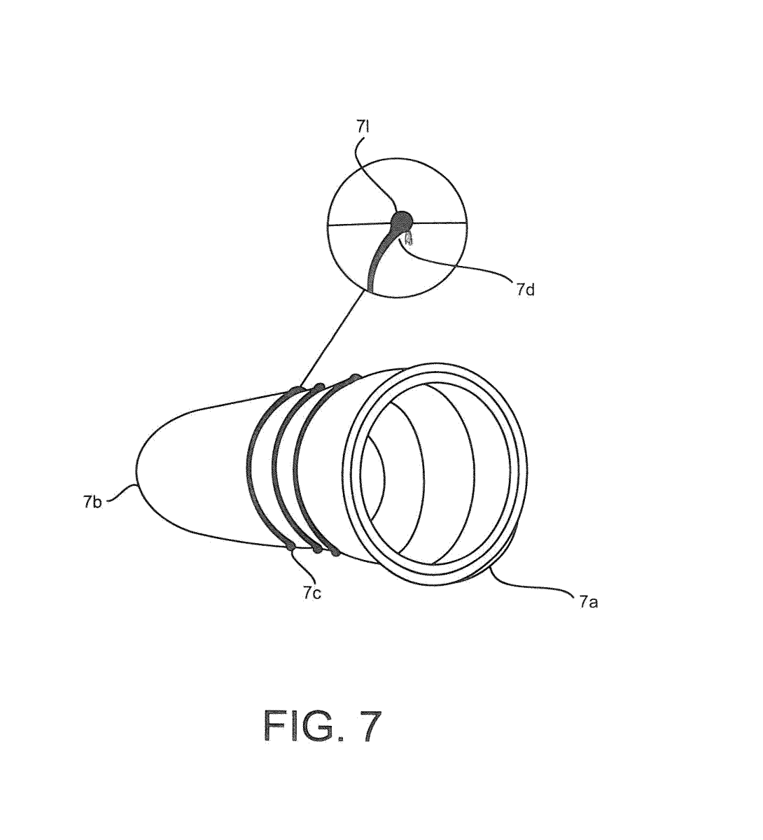

The goal of the above preferred iterations was to introduce a treatment into the male-side of the condom and retain it there. To help accomplish the patent further envisions that certain modification may be required to the condom itself (see FIG. 7). In addition to the iterations above, the bead end (7a) of the condom may be altered. The purpose of this alteration is an additional measure to further retard the migration of fluid treatment that was in the reservoir (7b) end and to further retard leakage of any fluid from the male-side.

A series of at least one internally (toward the penis) facing struts (7c) can be engineered to the dipping mandrel (used in manufacture of the condom, not shown in the drawings). In FIG. 7 a condom is shown with three struts (7c). These circumferential struts, cause by either engraving shaped, specially configured, indentations and/or extensions on mandrel formers, so that when dipped the latex adhered and was formed into one of more circumferential struts. These struts may have single or multiple tiers, they may be rounded or comma shaped (7d), and may be in any configuration that will enable the vulcanized rubber or elastomer substrate, once fully molded to be detached from the former. Additionally, the struts may be of a width and height of at least 1/16 inch and they may be uniformly shaped or have molded extensions to their peripheral configuration to that such extensions may protrude further in parts of the extension than at other parts of the extension. One example of the shape of the extension would be to create a continuous rib or ridge shaped like the aft part of a airplane wing (7e). To allow for penises of different diameters, the thickness (width) of any two such ribs or the amount that they protrude from the condom wall may be different.

Packaging for New Condom System Invention

Conventionally, male condoms are packaged automatically between at least two layers of foil and/or plastic laminate (which may be layered) and, after they are placed on to the continuous fed substrate, which is crimped and/or pressure/heat sealed, thus sandwiching the inserted condom. During the packaging and prior to sealing an amount of lubricant is added. This is the lubricant that migrates throughout conventional condom packages. The condom itself may or may not be self-oriented into a reservoir-end-up position. This results in confusion by the user as to the correct-way-on direction and often contributes to wrong-way-on donning and damage or tearing of the condom rendering it inefficient as a either a contraceptive, protective, or delivery device for a microbicide or spermicide.

When the user tears open the condom package just prior to use, that user is ordinarily given no sign or signal from the exterior of the package suggesting the correct-way-on for donning. This especially true in low light or dark conditions that ordinarily accompany the condom-use-time continuum. Therefore, when a prospective user who may be rushing to apply the condom, retrieves it from the conventional package, unless he or she looks closely, when the condom immediately is retrieved the condom has a 50% chance of being donned in the wrong direction. When this situation presents itself, if the condom is forced on by pressure it is likely to cover only a portion of the penis and worse, it is likely to tear and to unseat itself during coitus because of intromissive thrusting. Alternatively, the condom is removed, turned around, and rolled on correctly. Or, as is often the case, the act and time of removing it and re-orienting it, the concentration on that effort, and the real time that effort takes, often results in the loss of erection. Another result, one that becomes the most serious, yet very often used, solution is simply for the man to give up entirely on the condom itself and proceed with penetration without it. Of course, all protection is then eliminated in the pursuit of more instant gratification.

The present invention provides a solution for his problem, one which will also permit the selective and, in some iterations, automatic dosing of lubricant on either or both of the internal or external surfaces of the condom. Such dosing can be achieved by simply squeezing open an applicator that was packaged with the condom. Or, it can be achieved by pressure of the penis on a frangible pouch/pellet, as the condom is donned. Or it can be achieved simply by unsnapping a cover cap that had been put in place to cover the condom and its microbicide/spermicide contents. Or it can be achieved by twisting open a different applicator that would then be used to further don the condom accurately.

Regardless of which method, the end result will be a more easily, more speedily applied condom; one which was donned correctly, and one which contained a microbicide that covers the penis (when the condom is fully on) but had not leaked or migrated so as the contact the internal tissues of the female partner.

The present invention envisions three ridges separated from themselves and from the open end of the condom by varying spaces. Each individually and separately designed ridge, actually that will the have the effect of further restricting the return blood flow within the tissue immediately beneath the surface-most layer of the epidermal tissue of the shaft of the penis in that particular area. Their purpose is to constrict blood flow out from the penis resulting in maintenance of the erection.

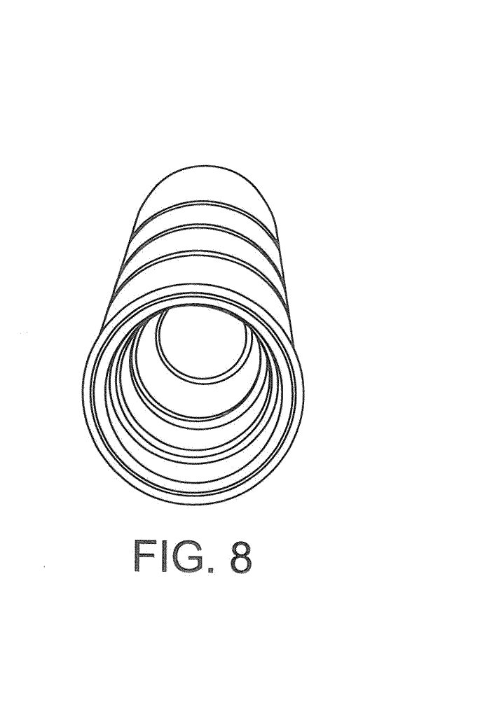

A further embodiment of the present invention combines a thin sponge-like material 8a (FIG. 8) that is oriented and may be applied and secured with latex compatible glue or may be set and held compressively between circumferential ridges on either side of the sponge-like material. The purpose of this material is to act as a further barrier and to absorb the medicinal fluid preventing it from leaking out of the open end of the condom. The sponge-like material would also be able to absorb semen.

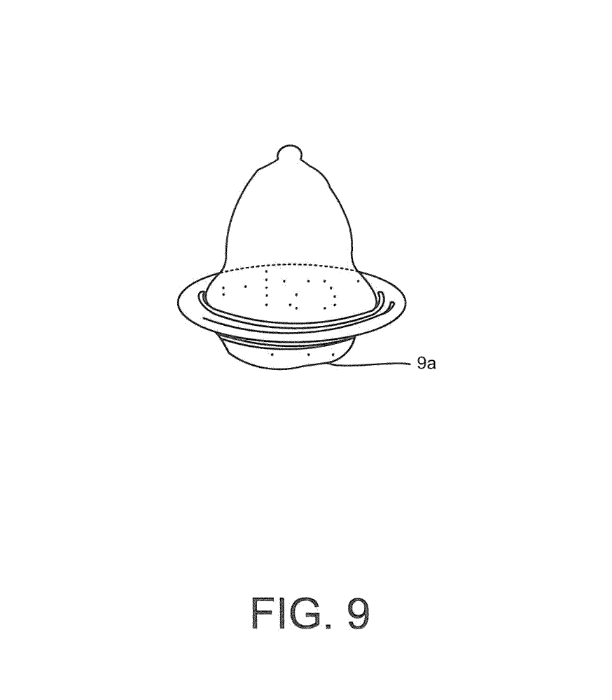

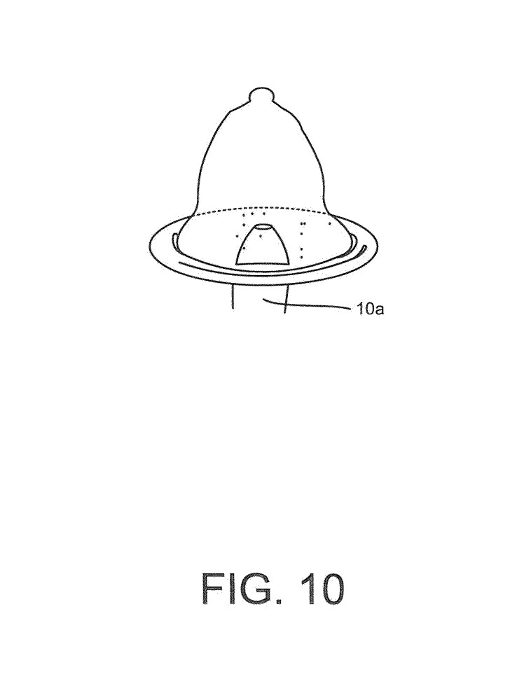

FIGS. 9 and 10 provide illustrations of the means of introducing the medicinal fluid to ordinary conventional condoms or additional lubricant to the embodiments within this patent. FIG. 9 envisions a frangible pouch (9a) that is attached to a foil seal, itself detachable attached to the rolled open end bead of the condom. Said pouch attachment is designed to fracture and disperse its semi pressurized contents the moment the seal at the open end of the condom is pulled open. Upon such fracture, the contents are then dispersed on the inside of the condom and further pushed to the reservoir tip area by the entry of the erect penis that acts as a piston in a tube. This action simultaneously covers the penis with the medicinal fluid as the condom is further rolled down the shaft.

FIG. 10 shows a similar configuration except that the users shall squeeze a pellet (10a) partially protruding from and held fact to the seal at the open end of the condom. The action of squeezing the frangible pellet fractures its pre-weakened end and sprays its semi-pressurized contents toward the reservoir tip end prior to being pulled from the open end of the condom. The contents are then further propelled toward the closed end and act to cover the penis as the erect penis is inserted into the condom and the balance of the condom is rolled down the shaft of the penis.

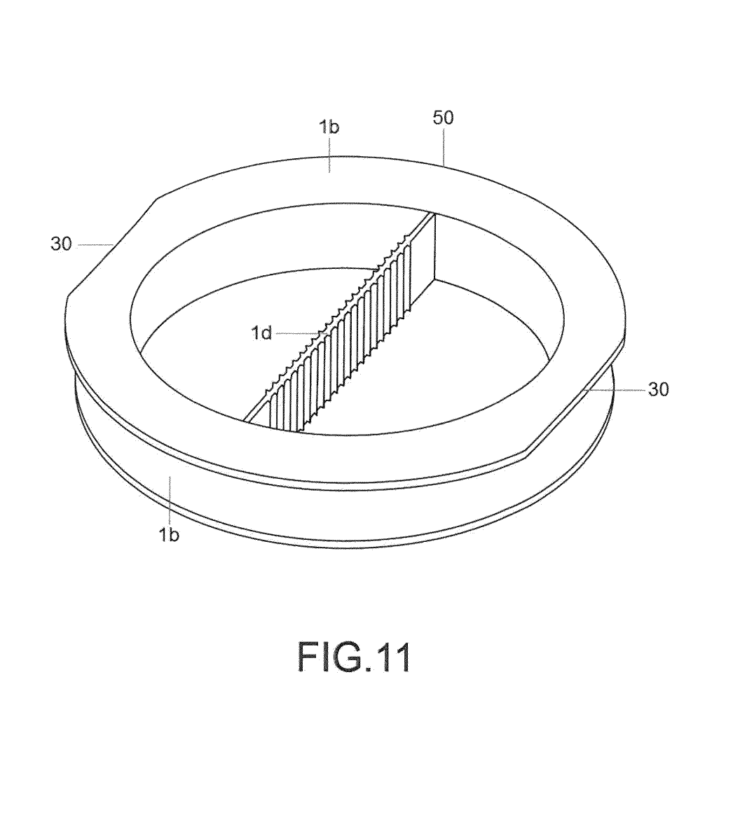

FIG. 11 shows an alternate embodiment of the invention. In this embodiment, a plug 50 is shown similar to the plug in FIG. 1. One difference is that the plug 50 has a rim 1c that is cut off at 30. This shape facilitates the installation of a condom on the rim 1b.

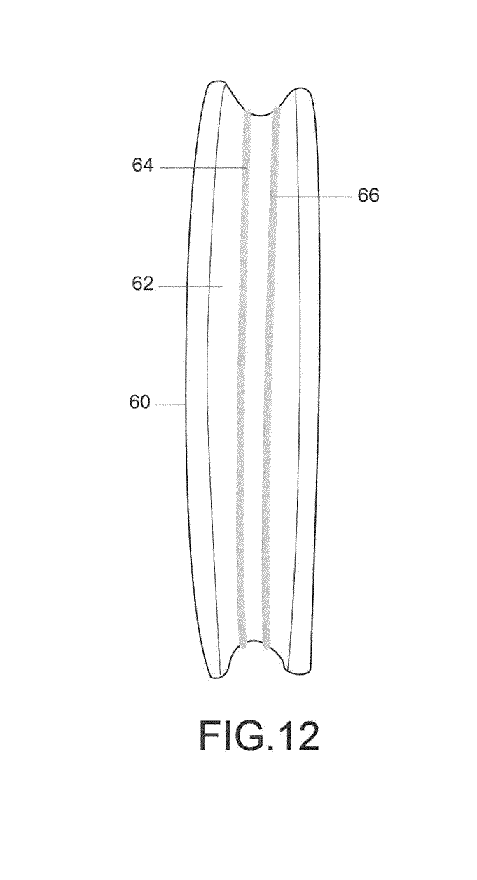

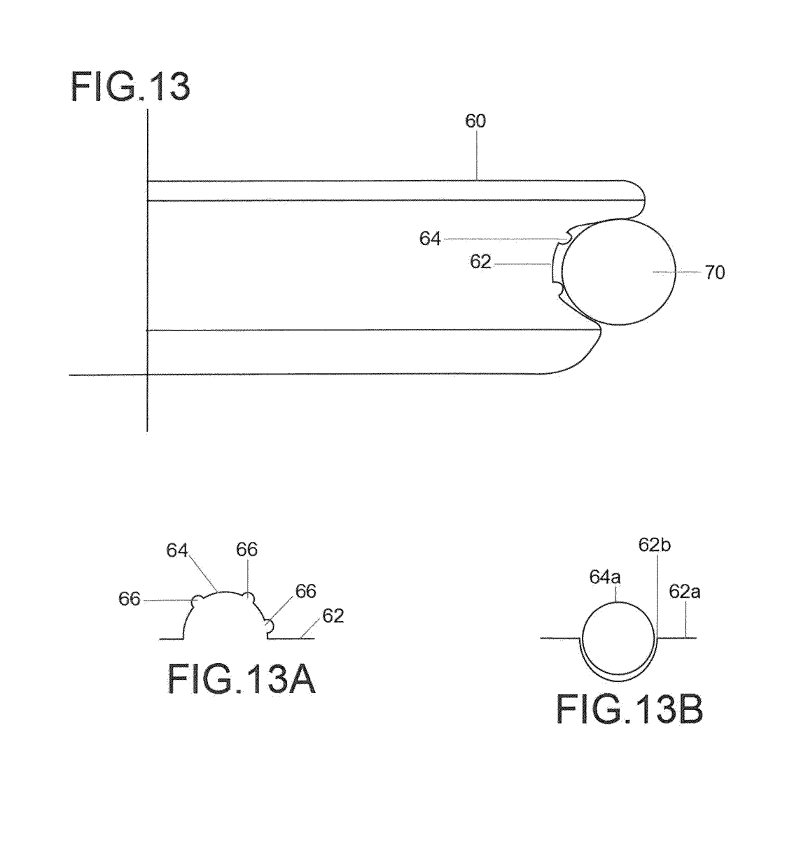

FIG. 12 shows another embodiment of the invention. In this embodiment, plug 60 is similar in shape to the plugs in FIGS. 1 or 11 however, its circumferential groove 62 is formed with two or more ridges 64. As shown in more detail in FIG. 13A, the ridges may have their own subridges 66.

In one embodiment, the ridges 64 and 66 being molded integrally with the plug 60.

In one embodiment shown in FIG. 13B, instead of an integral ridge, groove 62A is formed with one or more subgrooves 62B. O-rings 64A made of a flexible material and having a cross section equal to, or slightly smaller than the cross section of the subgrooves 62A are mounted in the groove 62A as shown. rO-rings 64A may have a generally circular cross section as shown or may be formed with subridges similar to the subridges 66 in FIG. 13A.

The purpose of the ridges 64, 66 and O-rings 64A is to form a secure, essentially air tight seal with the bead 70 of a condom.

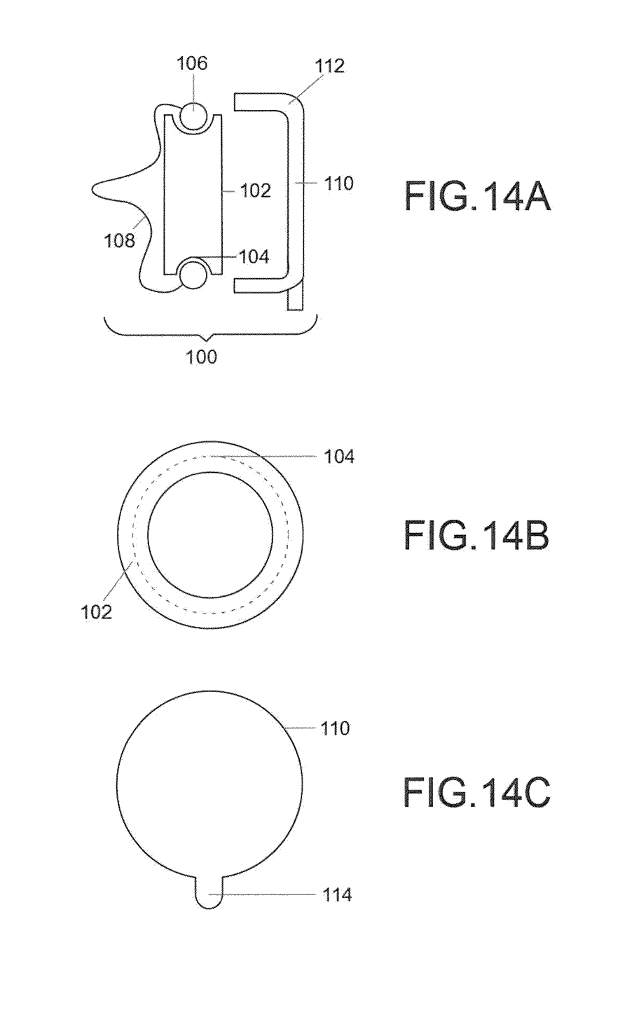

FIGS. 14A-14C show another embodiment of a condom assembly 100. In this embodiment, a condom assembly 100 includes a ring shaped plug 102 having a circumferential groove 104. The groove 104 is sized and shaped to receive the bead 106 of a rolled-up condom 108. The plug 102 can be made of plasticized paper or similar material. This material itself may be sufficient to provide a seal between the inner and outer surfaces of the condom 108, as discussed above. Alternatively, the groove 104 may be shaped to form subridges or provided with O-rings not shown) as discussed above in conjunction with FIG. 12. The assembly 100 further includes a semi-rigid cap having a generally circular shape, as shown in FIG. 14C. The cap 110 is formed with a rim 112 sized and shaped to snap over the plug 102 thereby trapping the bead 106 between the rim 112 and the groove 104. An extension or tail 114 is formed on the cap to assist in the removal of the cap 110 from the plug 102.

The assembly 100 is used as follows. The cap 110 normally forms a vacuum seal around the bead 106. Before use, the cap 110 is removed from the plug 102. Next, the plug 102 is squeezed diametrically inwardly so that the bead 106 can be easily removed from the groove 104. The plug 102 is then removed and discarded together with the cover 110 and the condom 108 is donned in the normal fashion.

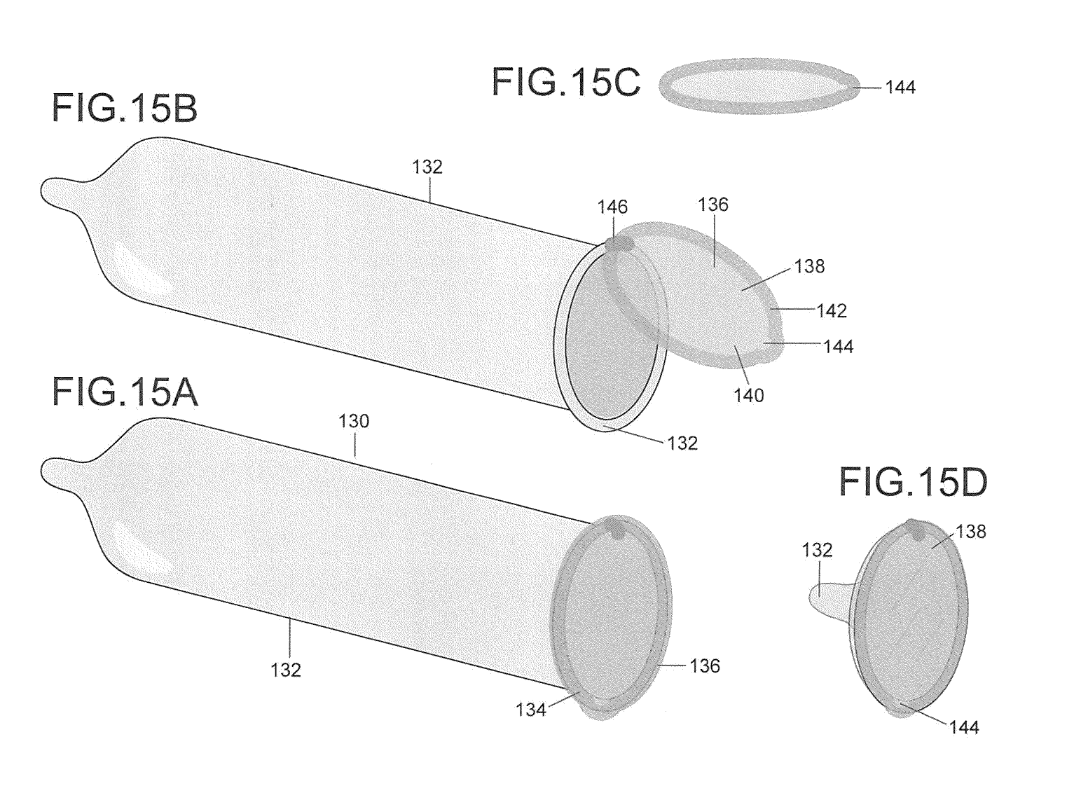

FIGS. 15A-15D show another embodiment of the invention. In this embodiment, a condom assembly 130 includes a standard condom 132 having a bead 134 and a cover 136. In FIGS. 15A and 15B the condom 132 is shown in an expanded configuration for the sake of clarity but it should be understood that a user never sees the assembly in this configuration, but instead, a user receives the condom assembly 130 with the condom 132 rolled up around its bead 134 as seen in FIG. 15D. The cover 136 is generally circular and made of a thin film made of silicone or other suitable material. The cover 136 can be transparent, translucent or opaque. The cover 136 includes an outer surface 138 and an inner surface 140. Around the circumferential edge of the inner surface 140 there is provided a ring-shaped adhesive layer 142. A pull tab is formed on the cover as at 144. In addition, a band 146 may be added to insure that the cover 136 does riot separate inadvertently from the condom. The cover is preferably about 0.001-003 in thick and made of a suitable plastic material.

The cover 136 is attached all around the bead 134 by the adhesive 144 and provides a seal that closes off the interior of the condom 132 and insures that there is no fluid exchange between the inner and outer surfaces of the condom as discussed above.

The condom assembly is packaged in the configuration shown in FIG. 15D. Before use, the cover 136 is peeled away from the bead 132 by pulling on tab 144. The cover 136 (including band 146) is then discarded.

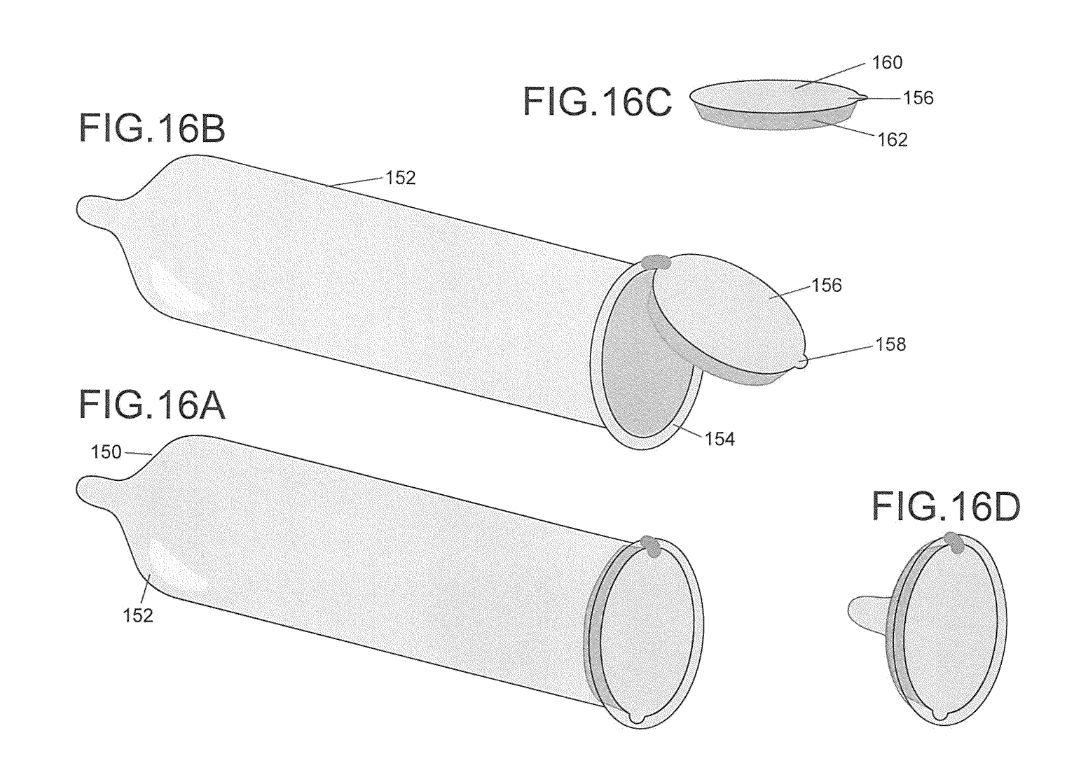

FIGS. 16A-16D shown another embodiment of the invention. This embodiment is very similar to the embodiment of FIGS. 15A-15D. Condom assembly 150 includes a cylindrical condom 152 with a bead 154 and a cover 156 seals off the inner and outer surfaces of the condom 152 as previously discussed. The cover 156 is made of a flexible or malleable plastic material that can be easily bent so that it can removed from the condom without damaging it. At one side, cover 156 has a pull tab 158 that can be used to remove the cover. An adhesive band 159 is used to secure the cover to the condom before use.

As shown in FIG. 16C, the cover 156 has a top surface 160 that is relatively flat and a lower portion 162 having a frustoconical or wedge shaped surface that allows the cover to be inserted into the bead 154 like a cork. Glue may be applied on portion 162 to maintain the cover in the bead 154. The cover 156 maybe hollow or solid. The rolled up condom package is shown in FIG. 16D.

Numerous modifications may be made to this invention without departing from its scope as defined in the appended claims.

What is claimed is:

1. A condom assembly comprising:
a plug having a disk shaped body with-an outer circumferential groove, said disk further including an upper surface; and
a condom having a bead disposed in said groove and a closed end portion extending across said upper surface.

2. The condom assembly of claim 1 further comprising a sealing element disposed in said groove and forming an airtight seal with said bead.

3. The condom assembly of claim 2 wherein said sealing element is a circumferential ridge formed on a toroidal wall of said groove.

4. The condom assembly of claim 3 said circumferential ridge includes circumferential subridges.

5. The condom assembly of claim 3 wherein said circumferential ridge is formed integrally with said plug.

6. The condom assembly of claim 2 wherein said sealing element is a circumferential O-ring disposed between a wall of said groove and said bead.

7. The condom assembly of claim 6 wherein said groove is formed with a circumferential subgroove receiving said O-ring.

8. The condom assembly of claim 6 wherein said O-ring is formed with circumferential subridges.

9. The condom assembly of claim 1 wherein said condom comprising:
an elongated body having a distal end that is closed, a proximal end that is opened to apply the condom on a penis, said cylindrical body having including an inner surface and an outer surface; and
a circumferential strut formed radially around said body at a predetermined distance from said proximal end, said strut being sized and shaped to prevent any liquid flow between said inner and outer surfaces while said condom is in use.

10. The condom of claim 9 wherein said strut is disposed at between 1″ and 2½″ from said proximal end.

11. The condom of claim 9 wherein said body is provided with up to three axially spaced struts.

12. The condom of claim 9 wherein said strut has a cross-sectional shape extending radially of said inner surface.

13. The condom of claim 9 wherein said strut has a generally v-shaped cross section.

14. The condom of claim 9 wherein said strut has the cross-section of an airplane wing.

15. The condom of claim 9 further comprising capsule of medicinal substance positioned to cause said medicinal substance to spread on said inner surface, with said strut preventing said medicinal substance from spreading to said outer surface.

16. The condom assembly of claim 1 wherein said condom comprising:
an elongated body having an inner surface, an outer surface, a distal end closed with a dome shaped tip, and a proximal open end, said elongated body having a generally frustoconical shape with said proximal end having a larger diameter then said distal end; and
a liquid barrier formed on said body, said liquid barrier being sized and shaped to prevent any liquid exchange between inner and outer surfaces.

17. The condom of claim 16 wherein said barrier is sized and shaped to prevent any liquid from flowing from said inner to said outer surface during the use of the condom.

18. The condom assembly of claim 1 wherein said plug has a bottom portion with a diametrically extending reinforcing member.

19. The condom assembly of claim 1 further comprising a cover attached to said plug to form an enclosure for said condom.

20. The condom assembly of claim 1 wherein said plug is made of cardboard.

21. A condom assembly comprising:
a condom having a generally cylindrical condom body with a closed end and an open end, with a circumferential bead formed at the open end; and
a cover having flat cover body, said cover being sized and shaped to cover said open end, said cover being adhesively attached to said bead to form a fluid seal with said bead.

22. The condom assembly of claim 21 wherein said cover is removably attached to said bead.

23. The condom assembly of claim 21 wherein said cover includes a tab shaped and sized for manual removal of said cover from said bead.
